(19) United States
(12) Patent Application Publication
(10) Pub. No.: US 2016/0090338 A1
(43) Pub. Date: Mar. 31, 2016

(54) METHODS FOR PRODUCING ALKYLAROMATICS

(71) Applicant: UOP LLC, Des Plaines, IL (US)

(72) Inventors: Kristy L. Geltz, Des Plaines, IL (US); Chad A. Williams, Arlington Heights, IL (US)

(21) Appl. No.: 14/864,168

(22) Filed: Sep. 24, 2015

Related U.S. Application Data
(60) Provisional application No. 62/055,073, filed on Sep. 25, 2014.

(51) Int. Cl. C07C 6/06

(52) U.S. Cl. CPC C07C 6/06; C07C 2529/08

(57) ABSTRACT

Disclosed is a method for process for transalkylation of aromatic compounds comprising introducing a feed stream comprising aromatic hydrocarbon compounds to the transalkylation zone; introducing a water source to the transalkylation zone; contacting the feed stream with a zeolitic transalkylation catalyst; and producing an ethylbenzene product stream. This method increases ethylbenzene yield while improving the selectivity of the catalyst.

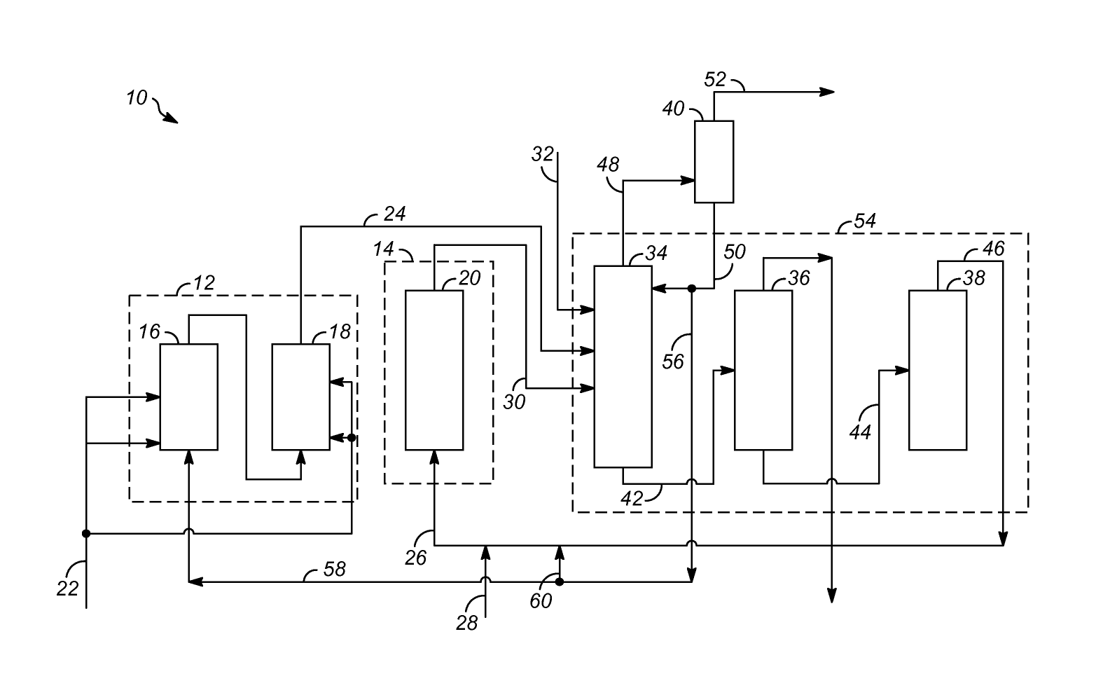

### CROSS-REFERENCE TO RELATED APPLICATION

This application claims priority from Provisional Application No. 62/055,073 filed Sep. 25, 2014, the contents of which are hereby incorporated by reference.

### FIELD

This subject matter relates to methods for producing alkylaromatics. More particularly, this subject matter relates to methods for producing alkylaromatics that may include introducing water to a transalkylation zone to increase ethylbenzene yield while improving the selectivity of the transalkylation catalyst.

### BACKGROUND

The alkylation or transalkylation of benzene with a C2 to C20 olefin alkylating agent or a polyaklyl aromatic hydrocarbon transalkylating agent is one of the primary sources for the production of alkyl-benzenes. For example, ethylbenzene is often produced by the alkylation of benzene with ethylene. Ethylbenzene may subsequently be used as a precursor for making styrene by the dehydrogenation of the ethylbenzene. Often, the ethylbenzene and styrene production facilities are integrated in an ethylbenzene-styrene complex so that after the ethylbenzene is produced it is sent to a downstream styrene plant that converts the ethylbenzene into styrene through dehydrogenation. Styrene may in turn be used to produce polystyrene, a widely used plastic, or other products.

In an alkyl-benzene production plant, benzene is fed along with a C2 to C20 olefin alkylating agent or polyalkylaromatic hydrocarbon transalkylating agent to an alkylation and/or transalkylation reactor. Typically, benzene is fed along with ethylene into an alkylation reactor, where alkylation of the benzene and ethylene over an alkylation catalyst forms ethylbenzene. The ethylbenzene product stream typically includes other components as well, such as poly-ethylbenzene. The stream may next be sent to a separation zone where the ethylbenzene is separated from other components in the stream to form a purified ethylbenzene stream. The poly-ethylbenzene stream is separated from other components and sent to a transalkylation zone where the poly-ethylbenzene is transalkylated with benzene to form additional ethylbenzene product. In an ethylbenzene-styrene complex, the ethylbenzene is next sent to a downstream styrene plant or zone of the complex for conversion of the ethylbenzene to styrene.

Catalysts for aromatic conversion processes generally comprise zeolitic molecular sieves. Examples include, zeolite beta (U.S. Pat. No. 4,891,458); zeolite Y, zeolite omega and zeolite beta (U.S. Pat. No. 5,030,786); X, Y, L, B, ZSM-5, MCM-22, MCM-36, MCM-49, MCM-56, and Omega crystal types (U.S. Pat. No. 4,185,040); X, Y, ultrastable Y, L, Omega, and mordenite zeolites (U.S. Pat. No. 4,774,377); and UZM-8 zeolites (U.S. Pat. No. 6,756,030 and U.S. Pat. No. 7,091,390).

It has been shown that water is not normally desired in the transalkylation zone for several reasons. First, as the water is introduced in to the transalkylation zone, the conversion of the desired product decreases. Second, as the water is introduced in to the transalkylation zone, the transalkylator catalyst activity decreases. In some cases, as the water input increases, and the desired product decreases, the operating inlet temperature must be increased in order to produce more of the desired product. The temperature would have to be increased in order to maintain the amount of desired product and the desired catalyst activity. However, by increasing the temperature to increase the amount of the desired product, a large portion of the delta temperature available for the catalyst life cycle is lost, which is why traditionally, water is not used as an input to a transalkylator.

However more recently, in order to remain competitive, the operating conditions in the alkylator have become more severe. Therefore, lower reactor temperatures and lower phenyl to ethyl ratios result in a lower alkylator selectivity and an increased flow to the transalkylator. Increased flow to the transalkylator often results in an increased yield loss. In an ethylbenzene production unit, the transalkylator is typically the main source of ethylbenzene yield loss. Accordingly, a need exists for a process that minimizes the loss of ethylbenzene in the transalkylator while maintaining or improving the transalkylator catalyst selectivity, as described and claimed herein.

### SUMMARY

This subject matter relates to methods for producing alkylaromatics. More particularly, this subject matter relates to methods for producing alkylaromatics that may include introducing water to a transalkylation zone to increase ethylbenzene yield while improving the selectivity of the transalkylator catalyst.

Hydrocarbon conversion processes, such as, for example, alkylation and/or transalkylation of a benzene feed stream to form ethylbenzene and the dehydrogenation of the ethylbenzene stream to form a styrene monomer stream are well known. Various aspects provided herein provide methods for adding a water injection stream into the transalkylation zone. The process to produce ethylbenzene from ethylene and benzene include two reactor sections: alkylation and transalkylation. Polyethylbenzenes produced from minor side reactions are recycled back to the transalkylation section and reacted with benzene to produce more ethylbenzene. The alkylator and transalkylator effluents are fractionated into recycle benzene, ethylbenzene product, recycle polyethylbenzene, and by-product flux oil typically streams using three distillations. In some designs, a fourth column, the light ends column, is used to remove a small amount of light ends, light non-aromatics and water from the recycle stream.

The benzene column recovers excess benzene from the reactor effluents. The recycle benzene stream for alkylator and transalkylator is typically obtained from the benzene column overhead. Benzene column bottom is fed to the ethylbenzene column where ethylbenzene product is recovered overhead. The ethylbenzene product is sent to the styrene section or to storage. Bottoms from the ethylbenzene column are fed to the polyethylbenzene column where polyethylbenzene is recovered overhead and recycled back to the transalkylator. The high boiling bottoms, flux oil, is cooled and sent to storage.

Although unsubstituted and monosubstituted benzenes, toluenes, and naphthalenes, are most often used, polysubstituted aromatics also may be employed. Examples of suitable alkylatable aromatic compounds in addition to those cited above may include anthracene, phenanthrene, biphenyl, xylene, ethylbenzene, propylbenzene, butylbenzene, pentylbenzene, hexylbenzene, heptylbenzene, octylbenzene, etc.; phenol, cresol, anisole, ethoxy-, propoxy-, butoxy-, pentoxy-, hexoxybenzene, and so forth. Sources of benzene, toluene, xylene, and or other feed aromatics include product streams from naphtha reforming units, aromatic extraction units, recycle streams from styrene monomer production units, and petrochemical complexes for the producing para-xylene and other aromatics. However, the hydrocarbon feed stream includes at least one aromatic hydrocarbon compound. According to one example, the concentration of the aromatic compound in the hydrocarbon feed stream ranges from about 5 to about 99.9 wt % of the hydrocarbon feed. By another example, the hydrocarbon feed stream comprises between about 50 and about 99.9 wt % aromatics, and may comprise between about 90 and about 99.9 wt % aromatics.

Here, the subject matter relates to methods for producing alkylaromatics. More particularly, the subject matter is a method for process for transalkylation of aromatic compounds comprising introducing a feed stream comprising aromatic hydrocarbon compounds to the transalkylation zone; introducing a water source to the transalkylation zone; contacting the feed stream with a zeolitic transalkylation catalyst; and producing an ethylbenzene product stream. This method increases ethylbenzene yield while improving the selectivity of the catalyst.

An advantage of the methods for producing alkylaromatics is that the ethylbenzene yield is improved once water is added to the transalkylator.

Another advantage of the methods for producing alkylaromatics is that the selectivity of the catalyst in the transalkylator is improved.

A further advantage of the methods for producing alkylaromatics is that the ethylbenzene yield is 99.5% to 99.9%.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWING

The FIGURE depicts one or more implementations in accord with the present concepts, by way of example only, not by way of limitations.

The FIGURE illustrates a flow diagram of an ethylbenzene production unit that includes a hydrocarbon feed stream alkylation zone and transalkylation zone having a water source in accordance with various embodiments.

### DETAILED DESCRIPTION

Hydrocarbon conversion processes, such as, for example, alkylation and/or transalkylation of a benzene feed stream to form ethylbenzene and the dehydrogenation of the ethylbenzene stream to form a styrene monomer stream are well known. Turning to the FIGURE, a flow diagram of an ethylbenzene production process is provided. Various aspects provided herein provide methods for adding a water injection stream into the transalkylation zone. The process to produce ethylbenzene from ethylene and benzene include two reactor sections: alkylation and transalkylation. Polyethylbenzenes produced from minor side reactions are recycled back to the transalkylation section and reacted with benzenes to produce more ethylbenzene. The alkylator and transalkylator effluents are fractionated into recycle benzene, ethylbenzene product, recycle polyethylbenzene, and by-product flux oil streams using three distillation columns. A fourth column, the light ends column, is used to remove a small amount of light ends, light non-aromatics and water from the recycle benzene stream.

The benzene column recovers excess benzene from the reactor effluents. The recycle benzene stream for alkylator and transalkylator is typically obtained from the benzene column overhead. Benzene column bottoms are fed to the ethylbenzene column where ethylbenzene product is recovered overhead. The ethylbenzene product may be sent to the styrene section, storage, or another location. Bottoms from the ethylbenzene column are fed to the polyethylbenzene column where polyethylbenzene is recovered overhead and recycled back to the transalkylator. The high boiling bottoms, flux oil, is cooled and may be sent to storage or another location.

The ethylbenzene production unit illustrated in the FIGURE includes a hydrocarbon feed stream 32, an alkylation zone 12, and a transalkylation zone 14 in accordance with various embodiments is provided. In the preferred process 10, the ethylene feedstock is fed via line 22 into the alkylation zone 12. The alkylation zone 12 in the example shown in the FIGURE includes a first alkylator 16 and a second alkylator 18. However, it is contemplated that in other embodiments there may be only one alkylator, or there may be more than two alkylators. As illustrated in the FIGURE, there are two alkylators, the first alkylator 16 and the second alkylator 18.

In an example, the first alkylator 16 includes a fixed bed reactor containing at least one bed of loose catalyst such as a zeolite, for example zeolite BEA (beta), zeolite MWW, zeolite Y, Mordenite catalyst, MFI catalyst, Faujasite catalyst, or any other molecular sieve catalyst suitable for liquid phase alkylation or combinations of any of the above catalysts. In the example shown in the FIGURE, a zeolite beta is preferred in the first alkylator 16. The first alkylator 16 operates in an adiabatic, liquid filled, single-phase mode. The first alkylator 16 may be an up-flow or a down-flow reactor. Up-flow is the preferred configuration. It is preferred that the first alkylator 16 operates in the temperature range of 180° C. to 270° C., a pressure of about 4.3 MPaG, and a typical liquid hourly space velocity (LHSV) in the range of 5.0 to 6.5 hr⁻¹, preferably around 5.2 hr⁻¹.

In an example, the second alkylator 18 is preferably a fixed bed reactor containing at least one bed of loose catalyst such as a zeolite, for example zeolite BEA (beta), zeolite MWW, zeolite Y, Mordenite catalyst, MFI catalyst, Faujasite catalyst, or any other molecular sieve catalyst suitable for liquid phase alkylation or combinations of any of the above catalysts. In the example shown in the FIGURE, a Zeolite beta is preferred in the second alkylator 18. The second alkylator 18 operates in an adiabatic, liquid filled, single-phase mode. The second alkylator 18 may be an up-flow or a down-flow reactor. Up-flow is the preferred configuration. It is preferred that the first alkylator 16 operates in the temperature range of 180 to 270, a pressure of about 4.3 MPaG, and a typical liquid hourly space velocity (LHSV) in the range of 5.0 to 6.5 hr⁻¹, preferably around 5.2 hr⁻¹.

The first akylator 16 may include a first alkylation catalyst and the second alkylator 18 may include a second alkylation catalyst. However, it is also contemplated that in another embodiment, the alkylation catalysts used in the first alkylator 16 and the second alkylator 18 may be the same.

By one aspect, the alkylator catalyst may include an acidic molecular sieve. Suitable acidic molecular sieves include the various forms of silicoaluminophosphates, and aluminophosphates disclosed in U.S. Pat. No. 4,440,871; U.S. Pat. No. 4,310,440 and U.S. Pat. No. 4,567,029 as well as zeolitic molecular sieves, which are incorporated herein by reference. As used herein, the term “molecular sieve” is defined as a class of adsorptive desiccants which are highly crystalline in nature, with crystallographically defined microporosity or channels, distinct from materials such as gamma-alumina. Preferred types of molecular sieves within this class of crystalline adsorbents are aluminosilicate materials commonly known as zeolites. The term “zeolite” in general refers to a group of naturally occurring and synthetic hydrated metal aluminosilicates, many of which are crystalline in structure. Zeolitic molecular sieves in the calcined form may be represented by the general formula:

Me_{2/n}O:Al₂O₃:xSiO₂:yH₂O

where Me is a cation, x has a value from about 2 to infinity, n is the cation valence and y has a value of from about 2 to 10. Typical well-known zeolites that may be used include chabazite, also referred to as Zeolite D, clinoptilolite, erionite, faujasite, Zeolite Beta (BEA), Zeolite Omega, Zeolite X, Zeolite Y, MFI zeolite, Zeolite MCM-22 (MWW), ferrierite, mordenite, Zeolite A, Zeolite P, and UZM-8 type zeolites referenced below. Detailed descriptions of some of the above-identified zeolites may be found in D. W. Breck, ZEOLITE MOLECULAR SIEVES, John Wiley and Sons, New York, 1974.

Significant differences exist between the various synthetic and natural materials in chemical composition, crystal structure and physical properties such as X-ray powder diffraction patterns. The molecular sieves occur as agglomerates of fine crystals or are synthesized as fine powders and are preferably tableted or pelletized for large-scale adsorption uses. Pelletizing methods are known which are very satisfactory because the sorptive character of the molecular sieve, both with regard to selectivity and capacity, remains essentially unchanged. In an embodiment, the adsorbent includes a Zeolite Y and/or Zeolite X having an alumina or silica binder and/or a beta zeolite having an alumina or silica binder. In an embodiment, the acidic molecular sieve is Zeolite Y.

In an embodiment, the molecular sieve will usually be used in combination with a refractory inorganic oxide binder. Binders may include either alumina or silica with the former preferred and gamma-alumina, eta-aluminum and mixtures thereof being particularly preferred. The molecular sieve may be present in a range of from 5 to 99 wt % of the adsorbent and the refractory inorganic oxide may be present in a range of from 1 to 95 wt %. In an embodiment, the molecular sieve will be present in an amount of at least 50 wt % of the adsorbent and more preferably in an amount of at least 70 wt % of the adsorbent.

The molecular sieve according to this example is acidic. Using silicon to aluminum ratio as a gauge for acidity level, the silicon to aluminum ratio should be no more than 100 in an embodiment and no more than 25 in a further embodiment. Cations on the molecular sieve are not desirable. Hence, acid washing may be desirable to remove alkali metals such as sodium in the case of Zeolite Y and Beta Zeolite to reveal more acid sites, thereby increasing the adsorptive capacity. Aluminum migrating out of the framework into the binder should also be avoided because it reduces acidity. Incorporation of some level of cations such as alkali earth and rare earth elements into Zeolite X or Y will improve the thermal and hydrothermal stability of the framework aluminum, minimizing the amount of framework aluminum migrating out of the framework, and may impart sites of varying acidic strength. The level of incorporation of the cations should be balanced to improve overall acidity and/or hydrothermal stability, without inhibiting adsorption performance that may result at higher cation incorporation levels. The molecular sieve adsorbent of the present subject matter may have the same composition as the alkylation catalyst in a downstream reactor, such as an alkylation or transalkylation unit. However, when the alkylation catalyst is more expensive than the molecular sieve adsorbent, the composition of the alkylation catalyst and the molecular sieve are preferably different.

A wide variety of catalysts can be used in the alkylation zone 12. The preferred catalyst for use in this subject matter includes a zeolitic catalyst. The catalyst of this subject matter will usually be used in combination with a refractory inorganic oxide binder. Preferred binders are alumina or silica. Suitable zeolites include zeolite beta described in U.S. Pat. No. 5,723,710, ZSM-5, PSH-3, MCM-22, MCM-36, MCM-49, MCM-56, type Y zeolite, and UZM-8, which includes the aluminosilicate and substituted aluminosilicate zeolites described in U.S. Pat. No. 6,756,030 and the modified UZM-8 zeolites, such as, UZM-8HS which are described in U.S. Pat. No. 7,091,390. Each of U.S. Pat. No. 6,756,030 and U.S. Pat. No. 7,091,390 is herein incorporated by reference in its entirety.

The basic configuration of a catalytic aromatic alkylation zone is known in the art. The feed aromatic alkylation substrate and the feed olefin alkylating agent are preheated and charged to generally from one to four reactors in series. Suitable cooling means may be provided between reactors to compensate for the net exothermic heat of reaction in each of the reactors. Suitable means may be provided upstream of or with each reactor to charge additional feed aromatic, feed olefin, or other streams (e.g., effluent of a reactor, or a stream containing one or more polyalkylbenzenes) to any reactor in the alkylation zone. The first alkylation reactor 16 and the second alkylation reactor 18 may contain one or more alkylation catalyst beds. Typically there are eight catalyst beds in series in an alkylation zone. The subject matter encompasses dual zone aromatic alkylation processes such as those described in U.S. Pat. No. 7,420,098 which is herein incorporated by reference in its entirety.

The alkylation reaction zone will often provide a wide variety of secondary by-products. For example, in the alkylation of benzene with ethylene to produce ethylbenzene, the reaction zone can also produce di- and triethylbenzene in addition to other ethylene condensation products. Another non-limiting exemplary reaction that is contemplated herein includes the alkylation of benzene with propylene to produce cumene. In this type of reaction, the reaction zone can produce di- and triisopropylbenzene in addition to still more condensation products. As is well known in the art, these polyalkylated aromatics may contact additional aromatic substrate in a transalkylation zone to produce additional monoalkylated product. See e.g. U.S. Pat. No. 7,622,622 and U.S. Pat. No. 7,268,267, which are incorporated by reference herein. In an embodiment, the alkylated benzene product comprises at least one of ethylbenzene and cumene.

An alkylated aromatic separation zone may also be provided for separating a concentrated alkylated aromatic stream from the alkylated aromatic stream produced by the alkylation zone 12. The alkylated aromatic separation zone 54 may include one or more distillation or fractionation columns or other separation apparatus as known in the art for separating a concentrated alkylated aromatic stream from other components in the alkylated aromatic stream. It should be noted that the term “concentrated” as used herein does not mean the resultant stream is free from other components, but rather that it has a higher concentration of the desired product than the stream fed into the separation apparatus. For example, as illustrated in the FIGURE, where the alkylation zone 12 produces an ethylbenzene stream via line 24, the alkylated aromatic separation zone may include an ethylbenzene separation zone 54 for separating a concentrated ethylbenzene stream from a stream including benzene, poly-ethylbenzene, and other components. A benzene fractionation column 34 may be in fluid communication with an outlet of the alkylation zone 12 and configured to receive the ethylbenzene stream via line 24 from the alkylation zone outlet and to separate benzene from the feed stream, which exits the benzene fractionation column through an alkylation benzene recycle stream via line 56. The alkylation benzene recycle stream may be passed back to the alkylation zone 12 as additional benzene feed. An ethylbenzene fractionation column 36 may be in fluid communication with the benzene fractionation column 34 via line 42 and may be provided to receive the benzene reduced ethylbenzene stream via line 42 to produce a concentrated ethylbenzene stream via fractionation. The ethylbenzene may provide a product stream or it may be transferred downstream. A poly-ethylbenzene fractionation column 38 may be provided to receive the ethylbenzene depleted stream via line 44 and to separate a concentrated poly-ethylbenzene stream, which may be recycled back to a transalkylation reactor 20 via line 46 as a feed to the transalkylation reactor to produce additional ethylbenzene.

The benzene recycle stream may be passed via line 56 back to the alkylation zone 12, via line 58 as shown in the FIGURE, where it is combined with the ethylene feed stream for treatment and subsequent alkylation of the combined benzene stream in the presence of ethylene to form additional ethylbenzene. The recycle benzene stream will first exit the benzene distillation column 34 via line 48 where it enters a lights removal column 40. The lights removal column 40 removes vent gas via line 52 and the remaining benzene exits out of the bottom of the lights removal column 40 via line 50 where it is recycled down line 56 and continues via line 58 to the alkylation zone 12, as shown in the FIGURE. A portion of the benzene recycle stream also will enter the transalkylation zone 14 via line 60.

The transalkylator 20 is preferably a fixed bed reactor containing at least one bed of loose catalyst such as a zeolite, for example zeolite BEA (beta), zeolite MWW, zeolite Y, Mordenite catalyst, MFI catalyst, Faujasite catalyst, or any other molecular sieve catalyst suitable for liquid phase transalkylation or combinations of any of the above catalysts. In the example shown in the FIGURE, a zeolite Y is preferred in the transalkylator 20. The transalkylator 20 operates in an adiabatic, liquid filled, single-phase mode. The transalkylator 20 may be an up-flow or a down-flow reactor. Up-flow is the preferred configuration. It is preferred that the transalkylator 20 operates in the temperature range of 190 to 245, a pressure of about 2.5 MPaG, and a typical liquid hourly space velocity (LHSV) in the range of 2.0 to 3.5 hr⁻¹, preferably at 3.0 hr⁻¹.

In the example shown in the FIGURE there is only one transalkylator 20. However, it is contemplated that there may be more than one transalkylator. For example, there may be two transalkylators that perform in series. In another example, there may be three or more transalkylators that perform in series.

In yet another example, the transalkylator catalyst may include an acidic molecular sieve. Suitable acidic molecular sieves include the various forms of silicoaluminophosphates, and aluminophosphates disclosed in U.S. Pat. No. 4,440,871; U.S. Pat. No. 4,310,440 and U.S. Pat. No. 4,567,029 as well as zeolitic molecular sieves, which are incorporated herein by reference. As used herein, the term “molecular sieve” is defined as a class of adsorptive desiccants which are highly crystalline in nature, with crystallographically defined microporosity or channels, distinct from materials such as gamma-alumina. Preferred types of molecular sieves within this class of crystalline adsorbents are aluminosilicate materials commonly known as zeolites. The term “zeolite” in general refers to a group of naturally occurring and synthetic hydrated metal aluminosilicates, many of which are crystalline in structure. Zeolitic molecular sieves in the calcined form may be represented by the general formula:

Me_{2/n}O:Al₂O₃:xSiO₂:yH₂O

where Me is a cation, x has a value from about 2 to infinity, n is the cation valence and y has a value of from about 2 to 10. Typical well-known zeolites that may be used include chabazite, also referred to as Zeolite D, clinoptilolite, erionite, faujasite, Zeolite Beta (BEA), Zeolite Omega, Zeolite X, Zeolite Y, MFI zeolite, Zeolite MCM-22 (MWW), ferrierite, mordenite, Zeolite A, Zeolite P, and UZM-8 type zeolites referenced below. Detailed descriptions of some of the above-identified zeolites may be found in D. W. Breck, ZEOLITE MOLECULAR SIEVES, John Wiley and Sons, New York, 1974.

Significant differences exist between the various synthetic and natural materials in chemical composition, crystal structure and physical properties such as X-ray powder diffraction patterns. The molecular sieves occur as agglomerates of fine crystals or are synthesized as fine powders and are preferably tableted or pelletized for large-scale adsorption uses. Pelletizing methods are known which are very satisfactory because the sorptive character of the molecular sieve, both with regard to selectivity and capacity, remains essentially unchanged. In an embodiment, the adsorbent includes a Zeolite Y and/or Zeolite X having an alumina or silica binder and/or a beta zeolite having an alumina or silica binder. In an embodiment, the acidic molecular sieve is Zeolite Y.

In an embodiment, the molecular sieve will usually be used in combination with a refractory inorganic oxide binder. Binders may include either alumina or silica with the former preferred and gamma-alumina, eta-aluminum and mixtures thereof being particularly preferred. The molecular sieve may be present in a range of from 5 to 99 wt % of the adsorbent and the refractory inorganic oxide may be present in a range of from 1 to 95 wt %. In an embodiment, the molecular sieve will be present in an amount of at least 50 wt % of the adsorbent and more preferably in an amount of at least 70 wt % of the adsorbent.

The molecular sieve according to this example is acidic. Using silicon to aluminum ratio as a gauge for acidity level, the silicon to aluminum ratio should be no more than 100 in an embodiment and no more than 25 in a further embodiment. Cations on the molecular sieve are not desirable. Hence, acid washing may be desirable to remove alkali metals such as sodium in the case of Zeolite Y and Beta Zeolite to reveal more acid sites, thereby increasing the adsorptive capacity. Aluminum migrating out of the framework into the binder should also be avoided because it reduces acidity. Incorporation of some level of cations such as alkali earth and rare earth elements into Zeolite X or Y will improve the thermal and hydrothermal stability of the framework aluminum, minimizing the amount of framework aluminum migrating out of the framework, and may impart sites of varying acidic strength. The level of incorporation of the cations should be balanced to improve overall acidity and/or hydrothermal stability, without inhibiting adsorption performance that may result at higher cation incorporation levels. The molecular sieve adsorbent of the present subject matter may have the same composition as the alkylation catalyst in a downstream reactor, such as an alkylation or transalkylation unit.

In one example, contacting conditions include a temperature of at least about 190° C. In the example shown in the FIGURE, there is water in the transalkylation zone 14 that enters via line 28. The presence of water in an amount of at least about 100 ppm relative to the hydrocarbon feed stream on a weight basis. Water may be present in an amount equal to or beyond the saturation point of the hydrocarbon feed stream at the contacting conditions. In an embodiment, water is present in an amount of at least about 100 ppm relative to the hydrocarbon feed stream on a weight basis. In another embodiment, water is present in an amount ranging from about 100 ppm to about 500 ppm relative to the hydrocarbon feed stream on a weight basis. In yet another example, water may be present in an amount ranging from about 300 ppm to about 500 ppm relative to the hydrocarbon feed stream on a weight basis. The amount of water during contacting may be controlled in any suitable manner. For example, the water content of the hydrocarbon feed may be monitored and controlled by drying and/or adding water or water generating compounds to the feed stream. Water or water generating compounds may be introduced as a separate stream to the contacting step, and the feed stream may be dried to a consistent water level while water or water generating compounds are added to obtain the desired content. In an example, the contacting temperature ranges from about 190° C. to about 245° C. and the contacting temperature may range from about 190° C. to about 230° C.

In an example, the amount of water is at least about 100 ppm relative to the hydrocarbon feed stream on a weight basis. In another example, the amount of water is at least about 500 ppm relative to the hydrocarbon feed stream on a weight basis. In another example, the amount of water equals or exceeds the saturation point of the hydrocarbon feed stream at the contacting conditions. For each of these examples, the contacting temperatures may include the ranges described in the immediately preceding paragraph. Optionally, the contacting conditions may further include a pressure around 3.0 MPa(g). In an example, the contacting is conducted with the feed in the liquid phase or partial liquid phase. In the example shown in the FIGURE, water is in the liquid phase. However, it is also contemplated that in alternative embodiments, water in the gas phase contacting may also be used.

A wide variety of catalysts can be used in the transalkylation zone 14. The preferred catalyst for use in this subject matter is a zeolitic catalyst. The catalyst of this subject matter will usually be used in combination with a refractory inorganic oxide binder. Preferred binders are alumina or silica. Suitable zeolites include zeolite beta described in U.S. Pat. No. 5,723,710, ZSM-5, PSH-3, MCM-22, MCM-36, MCM-49, MCM-56, type Y zeolite, and UZM-8, which includes the aluminosilicate and substituted aluminosilicate zeolites described in U.S. Pat. No. 6,756,030 and the modified UZM-8 zeolites, such as, UZM-8HS which are described in U.S. Pat. No. 7,091,390. Each of U.S. Pat. No. 6,756,030 and U.S. Pat. No. 7,091,390 is herein incorporated by reference in its entirety.

The basic configuration of a catalytic aromatic transalkylation zone is known in the art. The feed aromatic transalkylation substrate and the feed benzene transalkylating agent are preheated and charged to generally from one to four reactors in series. Suitable means may be provided upstream of or with each reactor to charge additional feed aromatic, feed olefin, or other streams (e.g., effluent of a reactor, or a stream containing one or more polyalkylbenzenes) to any reactor in the transalkylation zone. The transalkylator 20 may contain one or more alkylation catalyst beds. Typically there are 2 reactors in series in a transalkylation zone.

The transalkylation conditions usually include a pressure in the range between about 2.3 MPa(g) and 3.5 MPa(g). The transalkylation of the aromatic compounds with the olefins in the C2 to C20 range can be carried out at a temperature of about 190° C. to about 245° C. In a continuous process this space velocity can vary considerably, but is usually from about 2 to about 3.5 hr⁻¹ liquid hourly space velocity (LHSV) with respect to the olefin. In particular, the transalkylation of benzene with ethylene can be carried out at temperatures of about 190° C. to about 245° C. and the transalkylation of benzene with propylene at a temperature of about 100° C. to about 180° C. The ratio of transalkylatable aromatic compound to benzene used in the instant process will depend upon the degree of monoalkylation desired as well as the relative costs of the aromatic and benzene components of the reaction mixture. For transalkylation of polyethylbenzene by benzene, the Phenyl-to-Ethyl molar ratio may be as low as about 2.0 and as high as about 5.0. Where polyisopropylbenzene is transalkylated with benzene a Phenyl-to-Propyl ratio may be between about 1.5 and 4.0.

A transalkylated aromatic separation zone may also be provided for separating a concentrated transalkylated aromatic stream from the transalkylated aromatic stream produced by the transalkylation zone 14. As illustrated in the FIGURE, by one approach, the transalkylated aromatic separation zone and the alkylated aromatic separation zone may be a common zone or have common components. The transalkylated aromatic separation 54 zone may include one or more distillation or fractionation columns or other separation apparatus as known in the art for separating a concentrated transalkylated aromatic stream from other components in the transalkylated aromatic stream. It should be noted that the term “concentrated” as used herein does not mean the resultant stream is free from other components, but rather that it has a higher concentration of the desired product than the stream fed into the separation apparatus. For example, as illustrated in the FIGURE, where the transalkylation zone 14 produces an ethylbenzene stream via line 30, the transalkylated aromatic separation zone may include an ethylbenzene separation zone 54 for separating a concentrated ethylbenzene stream from a stream including benzene, poly-ethylbenzene, and other components. A benzene fractionation column 34 may be in fluid communication with an outlet of the transalkylation zone 14 and configured to receive the ethylbenzene stream via line 30 from the transalkylation zone outlet 30. An ethylbenzene fractionation column 36 may be in fluid communication with the benzene fractionation column 34 via line 42 and may be provided to receive the benzene reduced ethylbenzene stream via line 42 to produce a concentrated ethylbenzene stream via fractionation. The ethylbenzene may provide a product stream or it may be transferred downstream via line 44. A poly-ethylbenzene fractionation column 38 may be provided to receive the ethylbenzene depleted stream via line 44 and to separate a concentrated poly-ethylbenzene stream, which may be recycled back to a transalkylation reactor 20 via line 46 as a feed to the transalkylation reactor to produce additional ethylbenzene.

The benzene recycle stream may be passed via line 56 back to the alkylation zone 12, via line 58 as shown in the FIGURE, where it is combined with the ethylene feed stream for treatment and subsequent alkylation of the combined benzene stream in the presence of ethylene to form additional ethylbenzene. In the example shown in the FIGURE, the recycle benzene stream may first exit the benzene distillation column 34 via line 48 where it may enter a lights removal column 40. The lights removal column 40 removes vent gas via line 52 and the remaining benzene exits out of the bottom of the lights removal column 40 via line 50 where it is recycled down line 56 and continues via line 58 to the alkylation zone 12. A portion of the benzene recycle stream also will enter the transalkylation zone 14 via line 60.

The exemplary ethylbenzene production process illustrated in the FIGURE is intended to illustrate one possible process flow, and is not intended to limit the scope of the subject matter which may be practiced in other process flows. It is contemplated that in alternative embodiments, other configurations may be used.

A ⅝″ differential reactor was used to complete pilot plant testing on Y zeolite catalyst for the transalkylation of DEB with benzene at dry conditions and in the presence of water. The feed consisted of a mixture of 19.7% DEB/Benzene. The reactor feed moisture level was varied at 0, 82, and 529 ppm by weight. As water in the feed increased, inlet temperatures were increased to maintain approximately 80 weight percent DEB conversion. Online GC analysis of feed and product streams were completed every 6 hours. Additionally, liquid samples were collected and analyzed at the end of each testing condition to confirm the results of the online GC. Each testing condition was allowed sufficient time, greater than 40 hours at constant conditions, to allow for DEB conversion to stabilize. The results, as seen in the following Table, show at 82 ppm by weight water in the feed, inlet temperatures had to be increased by 5° C. to maintain DEB conversion at approximately 80 weight percent, compared to dry conditions. As water in the feed was increased further to 528 ppm by weight, the inlet temperature had to be increased 10° C. compared to dry conditions.

TABLE
0.7 hr⁻¹ DEB WHSV

| | | | |
|---|---|---|---|
| Water, wt ppm | 0 | 82 | 528 |
| DEB conversion, wt % | 79.9 | 79.6 | 79.1 |
| Inlet Temperature, ° C. | 195 | 200 | 205 |
| % Increase in Temperature | 0 | 12.5 | 25 |
| of (EOR T - SOR T) from | | | |
| 0 wt ppm water | | | |

The data demonstrates that traditionally the zeolite activity decreases when the water is increased in the benzene feed to the reactor. As shown in the above Table, as the water increases, the temperature must be increased as well in order to maintain the diethylbenzene conversion. The temperature would have to be increased because traditionally, as the water increases, the catalyst activity decreases. Therefore by increasing the water to 500 wt pm, 25% of the available temperature is lost, which is why traditionally, water is not used as an input to a transalkylator. Because of the decrease in catalyst activity, most unit designs allow for there to be minimal water in the transalkylator feed. Increased water in the recycle benzene feed to the alkylator can have a similar impact on alkylation catalyst activity by lowering the catalyst activity. Typically, when water is injected to the transalkylator, it is beneficial to remove the water in the transalkylator effluent through distillation to prevent the water from reaching the alkylator.

The above description and examples are intended to be illustrative of the subject matter without limiting its scope. While there have been illustrated and described particular embodiments of the present subject matter, it will be appreciated that numerous changes and modifications will occur to those skilled in the art, and it is intended in the appended claims to cover all those changes and modifications which fall within the true spirit and scope of the present subject matter.

1. A process for transalkylation of aromatic compounds comprising:
introducing a feed stream comprising aromatic hydrocarbon compounds to the transalkylation zone;
introducing a water source to the transalkylation zone;
contacting the feed stream with a transalkylation catalyst; and
producing an ethylbenzene product stream.

2. The process of claim 1 wherein the feed stream introduced to the transalkylation zone comprises benzene and polyethylbenzene.

3. The process of claim 1, wherein the water source is introduced to the transalkylation zone in an amount to provide between 100 ppm-wt and 500 ppm-wt of water based upon the mass of the feed stream.

4. The process of claim 1, wherein the zeolitic transalkylation catalyst comprises a modified Y zeolite catalyst.

5. The process of claim 1, wherein the transalkylation zone comprises at least one transalkylator.

6. The process of claim 1, wherein the ethylbenzene yield is 99.5 to 99.9% by weight.

7. A process for transalkylation of aromatic compounds comprising:
introducing a feed stream comprising aromatic hydrocarbon compounds to the transalkylator;
introducing a water source to the transalkylator;
contacting the feed stream with a zeolitic transalkylation catalyst in the presence of water; and
producing an ethylbenzene product stream.

8. The process of claim 7 wherein the feed stream introduced to the transalkylator comprises benzene and polyethylbenzene.

9. The process of claim 7, wherein the water source is introduced to the transalkylator in an amount to provide between 100 ppm-wt and 500 ppm-wt of water based upon the mass of the feed stream.

10. The process of claim 7, wherein the zeolitic transalkylation catalyst is a modified Y zeolite catalyst.

11. The process of claim 7, wherein there are two or more transalkylators.

12. The process of claim 7, wherein the ethylbenzene yield is 99.5 to 99.9% by weight.
